(19) United States
(12) Patent Application Publication
(10) Pub. No.: US 2025/0201355 A1
(43) Pub. Date: Jun. 19, 2025

(54) RETROSYNTHETIC TRANSLATION METHOD USING TRANSFORMER AND ATOMIC ENVIRONMENT, AND DEVICE FOR PERFORMING SAME

(71) Applicant: ARONTIER CO., LTD., Seoul (KR)

(72) Inventors: Ju Yong LEE, Gyeonggi-do (KR); Jun Su KO, Gyeonggi-do (KR)

(21) Appl. No.: 18/847,259

(22) PCT Filed: Mar. 13, 2023
(86) PCT No.: PCT/KR2023/003364

§ 371 Date: Sep. 15, 2024

(30) Foreign Application Priority Data
Mar. 17, 2022 (KR) 10-2022-0033648

(51) Int. Cl. G16C 20/30; G16C 20/70

(52) U.S. Cl. CPC G16C 20/30; G16C 20/70

(57) ABSTRACT

A novel retrosynthetic prediction method using fragment-based tokenization combined with transformer architecture is disclosed. Chemical reactions are represented using changes in a set of fragments of a molecule using an atom environment fragmentation scheme. An atom environment (AE) is an idealized and chemically meaningful component and generates a high-resolution molecular representation. Describing a molecule with a series of AEs establishes a clear relationship between translated product-reactant pairs due to the conservation of atoms in the reaction. A top accuracy of 67.1% within a biologically similar range on the USPTO test dataset is achieved, which outperforms other state-of-the-art translation methods. The impact of various encoding scenarios on the prediction of reactant candidates was investigated. A novel template-free model for retrosynthetic prediction provides fast and reliable retrosynthetic pathway planning for materials with distinct fragmentation patterns.

### TECHNICAL FIELD

The present invention relates to a retrosynthetic translation method using a transformer and an atom environment and a device for performing the same.

Retrosynthesis refers to a process of tracing through which pathway a target compound with a specific chemical structure is synthesized from any starting material in a synthetic reaction targeting an organic compound. To this end, the starting material is reached by identifying a precursor that may lead to the chemical structure of the target compound in a reverse direction of the actual reaction pathway, and then considering another precursor that reaches the precursor. This is an important step, especially in drug design, in virtually screening a group of candidates that can bind to a target protein, and then finding materials that can actually be synthesized or commercialized among these candidates.

### BACKGROUND ART

Planning a reaction pathway of an organic molecule is a key element of organic synthesis. The idea of reducing the complexity of desired organic molecules by considering all logical breaks forms the basis of a retrosynthetic approach. Therefore, the goal of the retrosynthetic approach is to propose a logical synthetic pathway to generate a target molecule from a series of possible reaction building blocks. The retrosynthetic approach recursively operates on the target molecule until a chemically plausible pathway is identified. From a broader perspective, predictors of forward and reverse reaction pathways in the literature may be divided into predictors that rely on the composition of reaction templates and data-driven networks trained in an end-to-end manner without templates.

A template-free method has emerged as an effective means of addressing the methodological limitations of the template-based paradigm. The template-free method may be further subdivided into (i) a graph-based method and (ii) a sequence-based method depending on the molecular representation method. The sequence-based modeling uses a molecular string representation to recast a reaction pathway planning problem as a language translation problem. Most of the current state-of-the-art forward and reverse response predictors are based on the transformer architecture (Document: [Ashish and Shazeer Vaswani Noam and Parmar, Attention is all you need, Adv. Neur. In. 2017-Decem (2017), no. Nips, 5999-6009, available at 1706.03762]). The transformer developed as a result of the collaboration is a neural machine translation (NMT) model that relies solely on an attention mechanism (Document: [Dzmitry and Cho Bahdanau Kyung Hyun and Bengio, Neural machine translation by jointly learning to align and translate, 3rd Int. Conf. Learn. Represent. ICLR 2015-Conf. Track Proc. (2015), 1-15, available at 1409.0473]). The molecular transformer was a first application of a transformer with a simplified molecular input line entry system (SMILES) applied to the task of predicting the forward reaction. Further studies have demonstrated its ability to perform general predictions using other compound databases, including drug-like molecules and carbohydrate reactions, to investigate regioselectivity and stereoselectivity. This success has paved a way for further studies on retrosynthesis using SMILES.

SMILES strings are a typical input for NMT models. Despite the widespread use of SMILES, SMILES is prone to making wrong predictions because of its grammatical complexity. That is, since SMILES-based prediction methods tend to make grammatically invalid predictions, prediction efficiency is reduced. To address this problem, SCROP (Document: [Shuangjia and Rao Zheng Jiahua and Zhang, Predicting Retrosynthetic Reactions Using Self-Corrected Transformer Neural Networks, Journal of Chemical Information and Modeling 60 (2020), no. 1, 47-55, DOI 10.1021/acs.jcim.9b00949]) included a neural network-based grammar corrector to reduce the invalidity rate. Similarly, Duan et al (Document: [Hongliang and Wang Duan Ling and Zhang, Retrosynthesis with attention-based NMT model and chemical analysis of “wrong” predictions, RSC Advances 10 (2020), no. 3, 1371-1378, DOI 10.1039/c9ra08535a]) focused on the cause of incorrect SMILES to improve prediction accuracy. In addition, grammatically valid SMILES is not guaranteed to be semantically valid or synthesizable. In this regard, it has been proven that representing molecules as a collection of fragments is an effective solution to the above-described problem (Document: [Daniel Lowe, Chemical reactions from US patents (1976-Sep2016), posted on 2017, DOI 10.6084/m9.gshare.5104873.v1]).

Given the complexity of retrosynthetic analysis, the efficient representation of source-target data structures is important for accurate prediction.

The embodiments of the present invention propose a direct translation approach for retrosynthetic prediction by associating the atom environment of reactants with products. The atom environment is a topological fragment centered on an atom with a preset radius (Document: [Hahnke, V. D., Bolton, E. E. & Bryant, S. H. PubChem atom environments. J. Cheminform. 7, 1-37 (2015) Mario and Hase Krenn Florian and Nigam, Self-referencing embedded strings (SELFIES): A 100% robust molecular string representation, Machine Learning: Science and Technology 1 (2020), no. 4, 045024, DOI 10.1088/2632-2153/aba947, available at 1905.13741]]). The radius is defined by the number of shortest topological distances between atoms via covalent bonds, i.e., the minimum number of covalent bonds. In the embodiment, these are considered as the basis of molecules and are used in a prediction workflow of the embodiment. The design of the embodiment helps capture the changes in molecules associated with the reaction by focusing on the fragments related to the reaction center. The transformer architecture, which has the best performance in NMT applications, is used to accurately generate the candidate reactants for the target molecule. The model of the embodiment shows that Top-1 accuracy is achieved with an accuracy of 53.4% for exact matches and an accuracy of 67.1% when including predictions that are biologically similar. These results are similar to or better than the existing methods without suffering from the problem of invalid predictions.

### DISCLOSURE

### Technical Problem

In retrosynthesis, since SMILES is grammatically complex, it is easy to make wrong predictions. That is, since the SMILES-based prediction methods tend to make grammatically invalid predictions, prediction efficiency is reduced. In addition, grammatically valid SMILES is not guaranteed to be semantically valid or synthesizable. The present invention is directed to providing results better than existing methods without suffering from the problem of invalid predictions.

### Technical Solution

According to one aspect of the present invention, a retrosynthetic translation method of predicting a reactant for a product using a neural machine translation (NMT) model based on transformer architecture includes: preparing an input sequence and an output sequence of the model, in which the input sequence and the output sequence represent molecules as a list of fragments, each fragment constituting the list of fragments is a fragment expressed based on an atom environment (AE), and the product and the reactant are converted into a sequence expressed as the AE and prepared as the input sequence and the output sequence, respectively; training the model through the input sequence and the output sequence; and predicting the reactant by retro-synthesizing the product through the trained model, in which a new product is converted into a sequence represented by the AE and input as an input sequence of the model, an output sequence is output through the model, and the predicted reactant is detected based on the output sequence, wherein the AE is a fragment composed of a central atom having a predetermined radius and its covalent neighbors, and the predetermined radius is a maximum allowable topological distance between the central atom and all covalent atoms.

The predetermined radius may be the number of bonds on a shortest pathway between atoms.

The fragment may be expressed as one of a set of AEs (AE0) with a predetermined radius of 0 and a set of AEs (AE2) with a predetermined radius of 1. The fragment may be expressed as a combination of a set of AEs (AE0) with a predetermined radius of 0 and a set of AEs (AE2) with a predetermined radius of 1.

The AE may be expressed as a simplified molecular-input line-entry system arbitrary target specification (SMARTS) pattern.

The SMARTS pattern for each of the AEs may be associated with a unique integer value.

The AE may be generated by an Extended Circular FingerPrint (ECFP) algorithm.

The model may use an encoder unit and a decoder unit, and apply a multi-head attention mechanism to each unit to translate the input sequence and the output sequence.

According to another aspect of the present invention, a retrosynthetic translation apparatus that predicts a reactant for a product using a neural machine translation (NMT) model based on transformer architecture includes a control unit controlling the NMT model; a communication unit communicating with an external server; a memory unit; a display unit; and an input unit receiving a user input, wherein the memory unit includes an input sequence and an output sequence of the model, the input sequence and the output sequence represent molecules as a list of fragments, each fragment constituting the list of fragments is a fragment expressed based on an atom environment (AE), and the product and the reactant are converted into a sequence expressed as the AE and stored as the input sequence and the output sequence, respectively, the control unit trains the model through the input sequence and the output sequence, the control unit converts a new product into a sequence represented by the AE for the trained model and inputs the sequence as an input sequence of the model, outputs an output sequence through the model, and detects a predicted reactant based on the output sequence, and the AE is a fragment composed of a central atom having a predetermined radius and its covalent neighbors, and the predetermined radius is a maximum allowable topological distance between the central atom and all covalent atoms.

### Advantageous Effects

According to an aspect of the present invention, an efficient representation of the source-target data structure for accurate prediction is disclosed, considering the complexity of retrosynthetic analysis. Specifically, embodiments of the present invention propose the direct translation approach for retrosynthetic prediction by associating the AE of reactants with products.

In addition, the design of the embodiment helps capture the changes in molecules associated with the reaction by focusing on fragments related to the reaction center.

### DESCRIPTION OF DRAWINGS

FIG. 1 is a schematic diagram of a model including an input-output structure according to an embodiment.

FIG. 2 is an example of a molecular representation according to an embodiment.

FIG. 3 is a histogram showing the number of Morgan bits according to the number of unique simplified molecular-input line-entry system arbitrary target specification (SMARTS) patterns according to an embodiment.

FIG. 4 is an example for evaluating the quality of a prediction according to an embodiment.

FIGS. 5A and 5B are diagrams illustrating representative examples belonging to each threshold level according to an embodiment.

FIG. 6 is a diagram illustrating the generation of AE0 and AE2 sets using all compounds of a dataset according to an embodiment and the visualization of diversity and coverage for a chemical space.

FIG. 7 is a diagram illustrating the results of a detection test according to an embodiment.

FIG. 8 is a schematic diagram of an apparatus for implementing a retrosynthetic method according to an embodiment.

### MODES OF THE INVENTION

The present invention will become apparent from the exemplary embodiments to be described below in detail together with the accompanying drawings in the present specification. However, the present invention is not limited to the embodiments disclosed below, and may be implemented in various different forms, these embodiments will be provided only in order to make the present invention complete and fully inform one of ordinary skill in the art to which the present invention pertains of the scope of the present invention, and the present invention will be defined by the scope of the claims. Meanwhile, terms used in the present specification are for explaining exemplary embodiments rather than limiting the present invention. Unless explicitly described to the contrary, a singular form includes a plural form in the present specification. Throughout this specification, the term “comprises” and/or “comprising” will be understood to imply the inclusion of stated constituents, steps, etc., but not the exclusion of any other constituents, steps, etc. Terms ‘first’, ‘second’, and the like, may be used to describe various components, but the components are not to be construed as being limited by these terms. The terms are used only to distinguish one component from another component.

### 1. Model Outline

The main goal of the transformer architecture is to generate the next word of a target sequence. The transformer uses encoder and decoder units to effectively apply a multi-head attention mechanism to each unit to translate between sequences. The input and output sequences for the transformer model are lists of fragments. The embodiment tested several different methods to transform molecules into a list of fragments, namely, a Molecular ACCess System (MACCS) key, a bit vector of Extended Circular FingerPrint (ECFP), and an atom environment (AE). The embodiment confirmed that an AE representation leads to the best model. The AE is a fragment composed of a central atom with a predetermined radius and its covalent neighbors. This may be considered as a basis for constructing a molecule, similar to a puzzle piece. Each AE is expressed as a simplified molecular-input line-entry system arbitrary target specification (SMARTS) pattern.

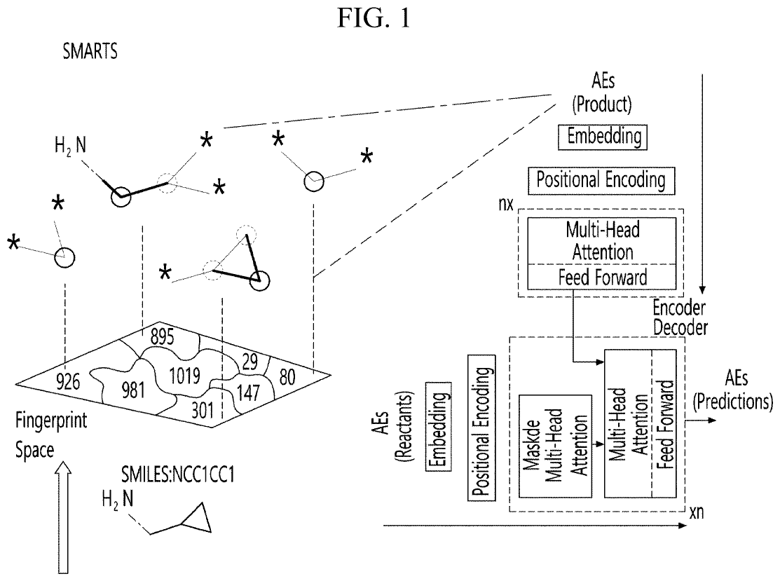

The outline of the transformer model of the embodiment, i.e., RetroTRAE, is described with reference to FIG. 1. Starting from the product molecule, it is decomposed into a set of unique integer values. The SMARTS patterns for each of the AEs are connected to a unique integer value. The list of AEs is provided as an input sequence of RetroTRAE. RetroTRAE is trained to predict the appropriate AE sequence for the reactant corresponding to the actual reactant.

### 2. Atom Environment

The embodiment uses the concept of a circular AE to represent the molecules in a reaction dataset. A circular environment is defined as topological neighborhood fragments of various ‘radii’ that include all bonds between the included atoms (Document: [Hahnke, V. D., Bolton, E. E. & Bryant, S. H. PubChem atom environments. J. Cheminform. 7, 1-37 (2015)]). They are centered on a specific atom called a central atom. The ‘radius’ refers to a maximum allowable topological distance between the central atom and all covalently bonded atoms. The topological distance between two atoms is measured as the number of bonds on the shortest path between the atoms. Therefore, an AE with a radius “r” includes all atoms of the molecule whose topological distance from the central atom is less than or equal to r and all bonds between the atoms.

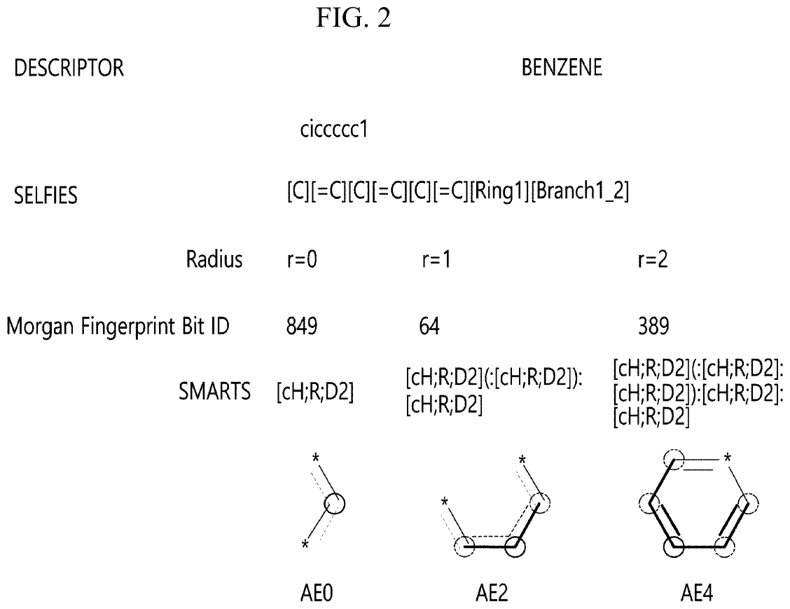

The embodiment used ECFPs of various radii implemented in RDKit to construct the AE. The embodiment extracted all unique fragments folded into bits of the ECFP. The AE generated by the ECFP algorithm is invariant to rotation and translation and may be easily interpreted as the SMARTS pattern as illustrated in FIG. 2. For example, an AE with a radius r=0 includes only the atom type of the central atom. The set of AEs with r=0 is represented by AE0. An AE with r=1 includes the central atom, all atoms (nearest neighbors) adjacent to the central atom, and all bonds between these atoms. The set of all AEs with r=1 is represented by AE2.

FIG. 2 shows a string representation of benzene as a combination of SMILES, self-referencing embedded strings (SELFIES), and SMARTS patterns generated by Morgan fingerprints. In the AE rendering, the central atom is shown in blue, and aromatic and aliphatic ring atoms are shown in yellow and gray. A wildcard [*] is used for all atoms. Specifically, the string representation of benzene in FIG. 2 is given by the generic SMILES and SMARTS patterns representing the AE generated by the ECFP fingerprints together with the recently developed SELFIES (Document: [Krenn, M., Ha “se, F., Nigam, A., Friederich, P. & Aspuru-Guzik, A. Self-referencing embedded strings (SELFIES): A 100% robust molecular string representation. Mach. Learn.: Sci. Technol. 1, 045024 (2020)]). SMARTS and SELFIES are very similar in the level of information displayed. A text part of the SMARTS representation includes two levels of details. The first detail relates to aromaticity and an H count of an element. The second detail includes the number of neighboring heavy atoms and ring information (represented by “D” and “R”, respectively). According to the definition, the environment of a radius of 0 corresponds to a single AE, all bits of radius 1 have at least 3 atoms, and each bit of radius 2 has at least 5 atoms.

The embodiment tested two fragmentation schemes, AEs and ECFPs. A word-based tokenization scheme is applied to the indices of both AE and ECFP bit vectors. The ECFP bit vector corresponds to a one-hot encoded vector in a fingerprint space, like a sentence, which is a one-hot encoded vector in a vocabulary space. The embodiment attempts the following representations encoded with the bit index and SMARTS:
AE0 and AE2 representing AEs with radius 0 and 1
ECFPO, ECFP2, and ECFP4 corresponding to Morgan fingerprints of radii of 0, 1, and 2-hashed to a dimension of 1024.

The AE (AE4) with radius 2 generates millions of distinct fragments in a large dataset. Due to the large vocabulary size of AE4, it is not suitable for translation purposes. Therefore, only the hashed version of the Morgan fingerprint is selected for radius 2. The open source RDKit module version 2020.03.1 is used to generate ECFPs and AEs.

### 3. Dataset

NMT methods require a large corpus of diverse source-target pairs for successful translation. To evaluate and compare the model of the embodiment with the current state-of-the-art, the present inventors used a subset of USPTO-Full, the filtered US patent reaction dataset obtained by the text mining approach. This subset includes 480,000 atom-mapped reactions after removing duplicate and incorrect reactions from USPTO-Full. The atom-mapped information was not used in training the model of the embodiment. However, an inherent benefit is gained from the fact that each atom in the product has a unique corresponding atom in the reactant. In addition, no reaction classification information is available in this dataset. The product-reactant pairs were curated to generate two distinct curated datasets composed of P→R and P→R_{A}+R_{B} type reactions having sizes of 100K and 314K, respectively. In addition, the present inventors also used the PubChem compound database including 111 million molecules and the ChEMBL database to recover molecules from the list of AEs list and compare the spaces of AEs.

### 4. Training Details

The curated dataset was randomly split at 9:1 to generate training and test sets. The validation set was randomly sampled from the training set (10%). The model parameters were trained using the stochastic gradient descent algorithm in conjunction with the negative log-likelihood (NLL) loss function. For each dataset, multiple tests were performed within the hyperparameter space range as described in Table 1 to achieve optimal performance. The optimal hyperparameters were selected based on the performance of the validation set. Using these hyperparameters, the average training speed corresponding to 1000 steps of a single reactant dataset was approximately 11 minutes per epoch. The present inventors trained the model for at least 1000 centuries using a learning rate scheduler stochastic gradient descent with warm restart (SGDR) and applied residual dropout with a rate of 0.1. The details of the hyperparameters are shown in Table 1 below.

TABLE 1

| | | Optimization model |
|---|---|---|
| Parameter | Possible value | parameter |
| Number of layers | 2-8 | 4 |
| Number of head | 4-12 | 8 |
| Size of hidden layers | 256, 512, 1024 | 512 |
| Size of intermediates | 512, 1024, 2048 | 2048 |
| Optimizer | Adam or SGD | SGD |
| Dropout | 0.1, 0.2, 0.5 | 0.1 |
| Number of epochs | 600-1500 | 1000 |
| Validation per epoch | @2-@100 | @100 |
| Learning Rate | 0.01-2.5 | 0.1, 0.05, 0.01 |
| Learning Rate Scheduler | Decay, SGDR | SGDR |
| Cycle per epoch | 3/1-1/3 | 5/4 |
| Decay factor | 0.8-0.98 | 0.91 |

### 5. Evaluation

To evaluate the performance of the translation model of the embodiment, an appropriate similarity measure should be selected to measure the similarity between the predicted and actual reactants. As two special cases of the Tversky index, the Tanimoto coefficient T_{c} and the Sørrensen-Dice coefficient S are indices selected for the embodiment. The exact form of the Tversky index is as follows.

$$S ( X , Y ) = \frac{\vert X ⋂ Y \vert}{\vert X ⋂ Y \vert + α \vert X - Y \vert + β \vert Y - X \vert} \qquad [ Equation 1 ]$$

Here, αₙβ≥O is a parameter of the Tversky index. When α=β=1 is set, it becomes the Tanimoto coefficient, and when α=β=0.5 is set, it becomes the Sørrensen-Dice coefficient. The Tanimoto and Dice coefficients measured between two molecules are between 0 and 1. A value of 0 represents total dissimilarity, and a value of 1 represents an exact match. The pairwise similarity between the predicted sequence and the correct sequence is calculated at the end of each epoch for all pairs present in the validation set using the selected metric.

Since there are many ways to decompose molecules, the retrosynthesis prediction tools may secure a large number of possible synthetic pathways. However, it is difficult to select an appropriate synthetic pathway. Based on the experience of the present inventors, the embodiment used the top-1 prediction as the best recommendation for reporting network performance as well as molecular search and detection. The embodiment used the ccbmlib Python package (Document: [Vogt, M. & Bajorath, J. Ccbmlib-A python package for modeling Tanimoto similarity value distributions. F1000Research 9, 100 (2020)]) to generate the similarity value distributions of the fingerprints and evaluate the statistical significance of the Tanimoto coefficients. In addition, this implementation allows quantitative comparison on similarity values between various fingerprint designs.

### 6. Performance of Embodiment

The present inventors evaluated the retrosynthesis predictor performance of the selected fingerprint variants to find the best molecular structure encoding. The results of the transformer model of the embodiment were compared with the previously developed substructure-based retrosynthesis predictors, as shown in Table 2.

TABLE 2

| | MIT-Single | | |
|---|---|---|---|
| Model | T_{C} = 1.0 | T_{C} = .85 | T̅_̅{̅C̅}̅ |

Bi-LSTM-based

| | | | |
|---|---|---|---|
| MACCS | 29.9 | 57.7 | 0.84 |
| ECFP2 | 35.6 | 50.7 | 0.80 |
| ECFP4 | 9.1 | 28.4 | 0.66 |

Transformer-based

| | | | |
|---|---|---|---|
| MACCS | 30.1 | 57.5 | 0.85 |
| ECFP0 | 50.8 | 61.2 | 0.85 |
| ECFP2 | 52.9 | 66.6 | 0.88 |
| ECFP4 | 26.0 | 50.1 | 0.73 |
| AE0 | 47.2 | 57.4 | 0.83 |
| AE2 | 50.9 | 59.9 | 0.84 |
| AE0 ∪ AE2 | 53.4 | 67.1 | 0.88 |

The transformer model representing molecules with a combination of AE0 and AE2 outperformed all other models by achieving an exact match accuracy of 53.4%. The relationship between structural similarity and biological activity has been extensively investigated in systematic analyses. It was found that molecules have similar biological activity when their similarity is greater than or equal to 0.85 (T_{c} ≥ 0.85). Adding biologically similar predictions (T_{c} ≥0.85) increases the accuracy 13.7% compared to the exact matches, so the overall model accuracy becomes 67.1%. Using ECFP2 also showed the good performance, but showed slightly lower performance than using AEs. Hereinafter, the combined model of AE0 and AE2 is referred to as RetroTRAE.

The transformer-based model showed significant improvements over the previous bi-LSTM-based methods in terms of exact match accuracy. This improvement represents an overall performance improvement of 15 to 17%. However, when MACCS keys were used for fragmentation, the number of exact matches and biologically similar matches was found to be similar. This suggests that the combination of MACCS keys may have limited diversity, i.e., low-resolution power. In contrast, AE2 provides a more precise description of the chemical space than MACCS keys and provides 60-fold higher resolution. This can be seen in Table 3 below.

TABLE 3

| | |
|---|---|
| Sequence length | Vocabulary size |

| Representation | Source | Target | Source | Target |
|---|---|---|---|---|
| MACCS | 32.30 | 39.15 | 130 | 131 |
| ECFP0 | 9.95 | 13.44 | 79 | 99 |
| AE0 | 9.95 | 13.44 | 119 | 118 |
| ECFP2 | 18.33 | 21.37 | 1025 | 1028 |
| AE2 | 18.33 | 21.37 | 7533 | 8007 |
| ECFP4 | 46.39 | 52.78 | 2052 | 2053 |

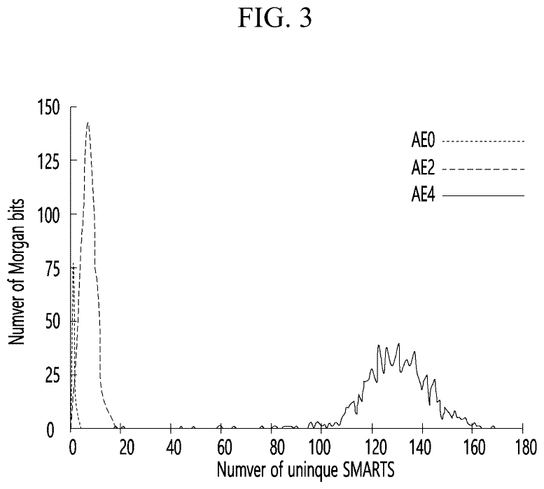

Another interesting observation is the poor performance of ECFP4. The number of exact matches is reduced to almost half that of ECFP2. The poor performance may be due to the high bit collision rate of ECFP4. FIG. 3 illustrates this. Specifically, FIG. 3 illustrates the histogram of the number of Morgan bits according to the number of unique SMARTS patterns from AE0 (blue), AE2 (green), and AE4 (red). As illustrated in FIG. 3, the present inventors investigated the number of unique AEs with radii 0, 1, and 2 associated with the activated bits of the hashed ECFP for the P→R type reaction data set. Each ECFP bit with radii 0 and 1 includes between 10 and 20 unique AEs. However, most bits with radius 2 correspond to many unique AEs between 100 and 160. That is, ECFP4 has a much higher bit collision rate than ECFP2 or ECFPO. The presence of high-density bits complicates the relationship between the fragments of the product and the actual reactants, which worsens the predictive power of the model. Therefore, finding the optimal set of fragments that most accurately represent the molecular structure is an important factor in improving the predictive power for retrosynthetic planning. The prediction performance as a function of different similarity thresholds for the best performing models is shown in Table 4. Table 4 shows the accuracy of single and dual reactant predictions using a combination of AE0 and AE2.

TABLE 4

| Dataset | T_{c} = 1.0 | SM | DM | T_{c} ≥ .85 | T_{c} ≥ .80 | T̅c̅ | S̅ |
|---|---|---|---|---|---|---|---|
| P--->R | 53.4 | 55.8 | 60.1 | 67.1 | 72.5 | 0.88 | 0.94 |
| P--->R_{A} + | 61.9 | 62.7 | 64.6 | 67.2 | 69.7 | 0.77 | 0.87 |
| R_{B} | | | | | | | |

It is indicated that single and double mutations representing one and two fragments do not match the observed data. In the embodiment, this is called a soft threshold. For the unimolecular reaction, the average reactant length of P→R is 27. The single and double fragment mutations correspond to T_{c}≥0.96 and T_{c}≥0.92. For the bimolecular reaction P→R_{A}+R_{B}, two reactants have an average length of 17. A detailed description of the similarity measure may be found in Table 5 below for the soft threshold as a function of reactant fingerprint length.

TABLE 5

| Length | 5 | 8 | 11 | 14 | 17 | 20 | 23 | 26 | 29 | 32 |
|---|---|---|---|---|---|---|---|---|---|---|
| T_{C} of SM | 0.80 | 0.88 | 0.91 | 0.93 | 0.94 | 0.95 | 0.96 | 0.96 | 0.97 | 0.97 |
| T_{C} of DM | 0.60 | 0.75 | 0.82 | 0.86 | 0.88 | 0.90 | 0.91 | 0.92 | 0.93 | 0.94 |

7. Comparison with Existing Retrosynthetic Planning Models

Table 6 compares the performance of the available retrosynthetic models trained without reaction class information with the model of the embodiment. For a fair comparison, the present inventors compared RetroTRAE with the models trained and tested with the MIT-full USPTO dataset. The approach of the embodiment achieves the top-1 exact match accuracies of 53.4% and 61.9% for unimolecular and bimolecular reactions without reaction class information (Table 6). In general, this accuracy level is superior to most existing non-transformer and transformer models. The performance of RetroTRAE may compare with the highest level of the existing method, i.e., Lin's Transformer model. When considering biologically similar predictions, the overall accuracy on both datasets increases to 67.1%. This result significantly outperforms all the current state-of-the-art approaches.

TABLE 6

| | |
|---|---|
| | Top-1 |
| Model | accuracy (%) |

Non-Transformer

| | |
|---|---|
| Coley et al., Similarity, 2017 | 32.8 |
| Segler et al., Neuralsym, 2017 | 35.8 |
| Segler-Coley,-rep. by Lin, 2020 | 47.8 |
| Dai et al., GLN, 2019 | 39.3 |
| Liu et al.-rep. by Lin, 2020 | 46.9 |

Transformer-based

| | |
|---|---|
| Zheng et al., SCROP, 2020 | 41.5 |
| Wang et al., RetroPrime, 2021 | 44.1 |
| Tetko et al., AT, 2020 | 46.2 |
| Lin et al., 2020 | 54.1 |
| RetroTRAE − Embodiment of the present invention | 53.4 |
| RetroTRAE + Bioactive − Another embodiment of the | 67.1 |
| present invention | |

Coley et al., Similarity, 2017 [Document: Coley, C. W., Rogers, L., Green, W. H. & Jensen, K. F. Computer-assisted retrosynthesis based on molecular similarity. ACS Cent. Sci. 3, 1237-1245 (2017).]
Segler et al., Neuralsym, 2017 [Document: Segler, M. H. & Waller, M. P. Neural-symbolic machine learning for retrosynthesis and reaction prediction. Chem.-A Eur. J. 23, 5966-5971 (2017).]
Segler-Coley,-rep. by Lin, 2020 [Document: Lin, K., Xu, Y., Pei, J. & Lai, L. Automatic retrosynthetic route planning using template-free models. Chem. Sci. 11, 3355-3364 (2020).]
Dai et al., GLN, 2019 [Document: Dai, H., Li, C., Coley, C. W., Dai, B. & Song, L. Retrosynthesis prediction with conditional graph logic network. Adv. Neural Inform. Process. Syst. 32, 1-11 (2019).]
Liu et al.-rep. by Lin, 2020 [Document: Lin, K., Xu, Y., Pei, J. & Lai, L. Automatic retrosynthetic route planning using template-free models. Chem. Sci. 11, 3355-3364 (2020).]
Zheng et al., SCROP, 2020 [Document: Zheng, S., Rao, J., Zhang, Z., Xu, J. & Yang, Y. Predicting retrosynthetic reactions using self-corrected transformer neural networks. J. Chem. Inf. Model. 60, 47-55 (2020).]
Wang et al., RetroPrime, 2021 [Document: Wang, X. et al. Retroprime: A diverse, plausible and transformer-based method for single-step retrosynthesis predictions. Chem. Eng. J. 420, 129845 (2021).]
Tetko et al., AT, 2020 [Document: Tetko, I. V., Karpov, P., Van Deursen, R. & Godin, G. State-of-the-art augmented NLP transformer models for direct and single-step retrosynthesis. Nat. Commun. 11, 1-11 (2020).]
Lin et al., 2020 [Document: Kangjie and Xu Lin Youjun and Pei, Automatic retrosynthetic route planning using template-free models, Chemical Science 11 (2020), no. 12, 3355-3364, DOI 10.1039/c9sc03666k]

The average T_{c} of the predictions by the best performing model is 0.88, which is statistically highly significant with a p-value<10-5 (Table 4).

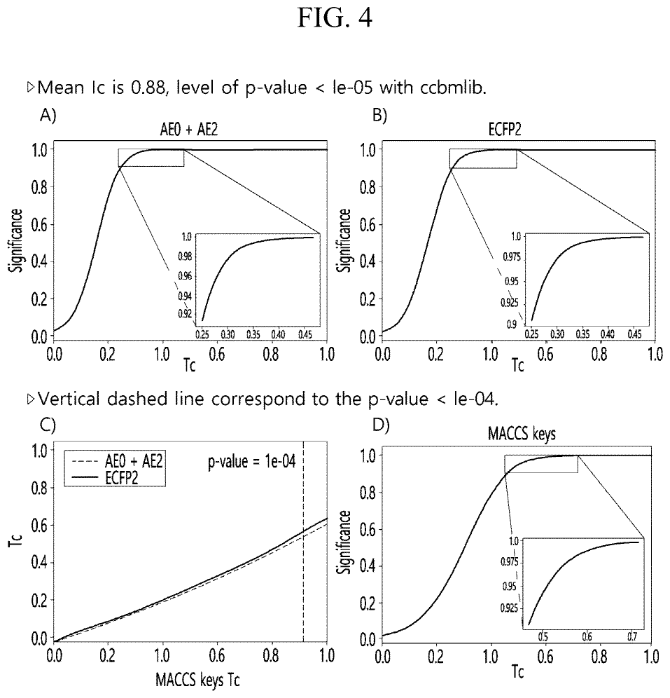

FIGS. 4A, 4B, and 4D illustrate the cumulative distribution functions of the reactants in the USPTO database for the integrated AE, ECFP2, and MACCS keys, respectively. The measure 1-(p-value) is used to evaluate significance. The p-value ranges from 0 to 1, and the smaller the p-value, the higher the significance. FIG. 4C illustrates the relationship between a MACCS keys T_{c} value and a T_{c} value for the integrated AE and ECFP2. The vertical dashed line corresponds to a significance level of the p-value set to 1e-04. FIG. 4 illustrates the statistical significance of the selected similarity thresholds that may be used to evaluate the quality of non-identical predictions in chemical terms. The inset of the drawing shows a regime where the T_{c} value has a p-value of 0.1, but the lowest similarity threshold (T_{c} >0.8) in the embodiment has a p-value of 1e-04 or lower. Therefore, it may be said that predictions satisfying T_{c} >0.8 occur under high similarity conditions. The statistical equality between the similarity scores of each fingerprint type used in the embodiment is as illustrated in FIG. 4C. The integrated AE and ECFP2 share similar distribution profiles (see FIGS. 4A and 4B). Therefore, the embodiment shows that they return almost the same similarity values as presented in FIG. 4C. The vertical dashed line corresponds to the p-value 1e-04. Landrum (Document: [RDKit: Open-Source Cheminformatics Software http://www.rdkit.org (2016).]) showed that only 250 out of 25,000 pairs have Tanimoto similarity values greater than 0.434, and when computed with ECFP2 and MACCS keys, have Tanimoto similarity values greater than 0.655. Similarly, the lowest similarity threshold T_{c}>0.8 in the embodiment corresponds to T_{c} >0.9 when computed with MACCS keys.

### 8. Example of Biological Similarity Prediction

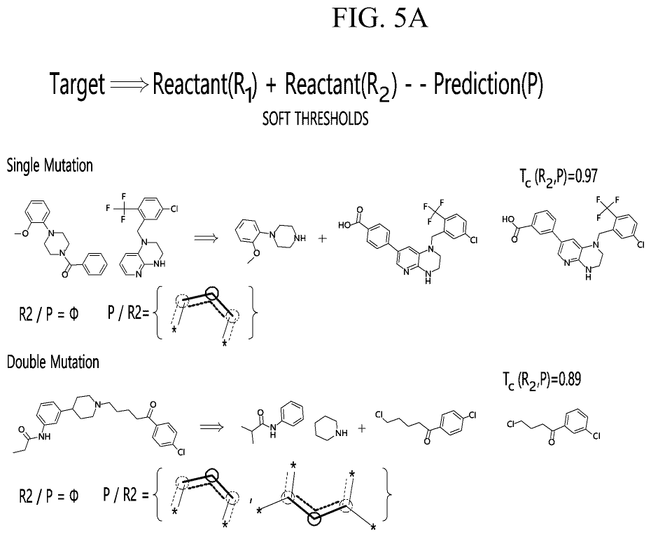

Similarity scores are a feasible and effective measure for evaluating the quality of retrosynthetic predictions. Therefore, single and double fragment mutations, bioactive, and highly similar predictions were included as high-quality reactant candidates. FIG. 5 illustrates representative examples of each category. Specifically, FIG. 5 illustrates representative examples belonging to each threshold level. The distinct fragments are provided as SMARTS patterns. The predictions are plotted as a similarity map using the Morgan fingerprints. The first reactant is accurately predicted, and the quality of the second reactant is evaluated. The fragments that are only part of the prediction and their actual counterparts may be marked differently to more specifically describe the chemical change. Colors indicate atomic-level contributions to the overall similarity (green: increased similarity score, red: decreased similarity score, colorless: no effect).

These examples are not identical, but help to chemically interpret biologically similar predictions. In the case of single mutations, the changes are often related to misplacement of functional groups in the ortho/meta/para positions. In the case of double mutations, most changes were observed in the ortho/meta/para substitution patterns, similar to the single mutation cases. Furthermore, a length of a simple aliphatic chain is often incorrectly predicted because many fragments of a long aliphatic chain are identical. Therefore, the length of an aliphatic chain may not be accurately described by a unique set of fragments. As can be seen in the similarity map, no atom in the reactant candidates contributes negatively to the similarity value. As a result of examining biological activity similar predictions, it was concluded that the most important aspects of retrosynthetic analysis, such as bond disconnections, reactive functional groups, and core structures, were correctly predicted. In the embodiment, the number of AEs changed when using hard thresholds may be two or more. However, these are mainly observed in the core structures and do not affect the accuracy of the reactive sites.

9. Interpretation of Chemical Space through AE

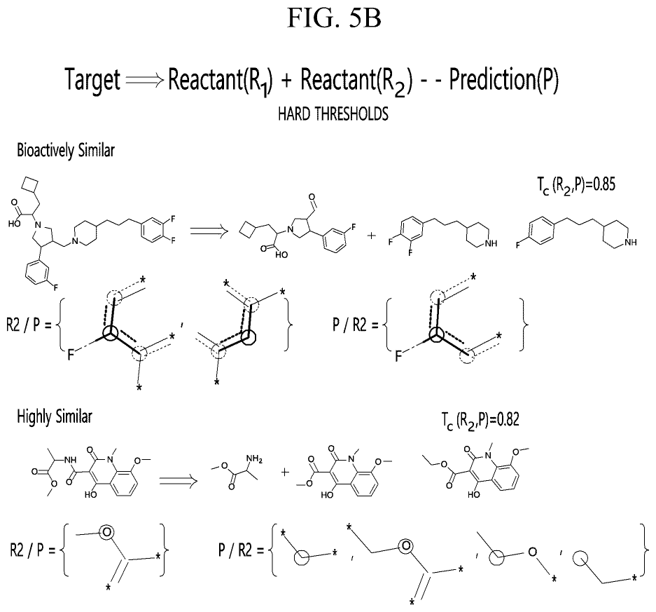

As described above, AEs may be considered as the basis of molecules. By using all compounds from the PubChem (111M), ChEMBL (2.08M), and USPTO 500K (1.3M) datasets, AE0 and AE2 sets were generated, and the diversity and coverage of the chemical space were visualized (FIG. 6). The area-proportional Euler graph (FIG. 6) demonstrates that the AEs of the reactants in the USPTO dataset do not span a broad chemical space. The USPTO reaction dataset includes 275 (r=0) and 15,982 (r=1) unique AEs. ChEMBL and PubChem include 386 (r=0), 39,149 (r=1) and 3,450 (r=0), 533,276 (r=1) unique AEs, respectively. Although PubChem has many more AEs, most of the AEs occur at a very low probability. In fact, many AEs in PubChem are found in only one compound, which is referred to as a singleton in the embodiment. The percentage of singletons is 38.5% and 35.2% for the AE0 and AE2 sets generated from the PubChem.

10. Detection of Molecules from AEs

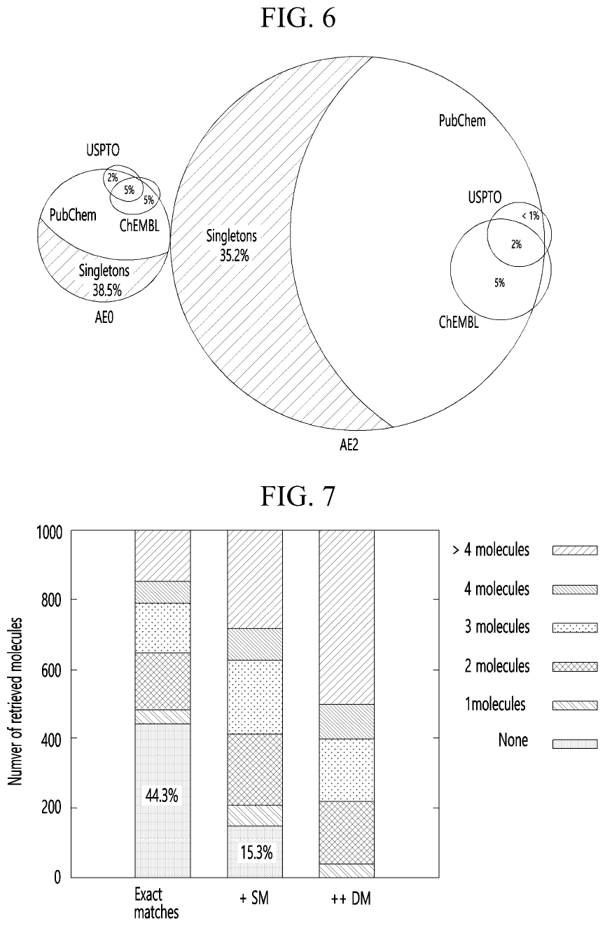

After the prediction is performed by RetroTRAE in the embodiment, the chemical structures of the predicted reactants may be detected through a database search. The embodiment investigated the success rate of detecting reactant candidates with 1,000 USPTO test molecules using PubChem. The detection test results showed that more than half (55.7%) of the predictions may be accurately detected (FIG. 7). Allowing single mutations increases the detection rate 30%. Allowing double mutations may successfully detect all test molecules. These results suggest that representing and predicting molecules as fragments is a feasible and practical approach.

Considering the degeneracy of fragment representation, using the Top-1 prediction does not necessarily lead to a single synthetic pathway. It is always possible to access multiple candidates in the process of converting fragments into valid molecules. This may correspond to multiple possible reaction pathways. Considering the small differences between molecules with a high T_{c} value (FIG. 5), many molecules generally differ in stereochemistry, the length of the aliphatic chain, and the positions of peripheral functional groups such as ortho/meta/para positions. Therefore, these small differences may be easily corrected by experienced chemists.

In addition, it is worth mentioning that AE is less degenerate than ECFP fingerprints in the detection process. Using ECFP bit indices for database searches, 1.7 times more reactive candidates are detected on average. The difference is mainly due to bit collisions and the absence of stereochemical information in the dataset.

### 11. Conclusions

The embodiment discloses a novel template-free retrosynthetic prediction model, RetroTRAE, using the transformer architecture and AE representation. RetroTRAE shows similar or improved performance compared to other state-of-the-art models. The current approach provides an exact match accuracy of 53.4% for the reactant candidates. In addition to the exact match, the high-quality reactant candidates selected by soft thresholds and hard thresholds are statistically significant at the 1.0e-04 level or lower. The average prediction accuracy with threshold T_{c}=0.85 is about 67%, which significantly outperforms the current state-of-the-art methods. The AE has proven to be a promising descriptor for studying reaction pathway prediction and discovery because it provides a highly descriptive representation without the grammar complexity of SMILES.

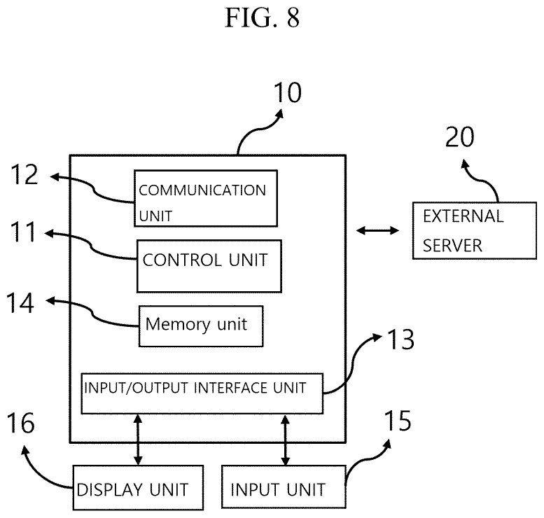

FIG. 8 is a block diagram illustrating an example of a system for implementing a retrosynthetic prediction method in an embodiment, and conceptually shows parts related to the embodiment. Each component may be equipped in one device and perform processing separately, but is not limited thereto, and may be connected through a network and may also be performed on a separate device.

An external server 20 may be connected to a prediction system 10 through a network, and may provide information on the reactant and product pairs, information on the AE of molecules, etc. For example, the external server 20 may include a large corpus of information on various source-target pairs, and for this purpose, may use a subset of USPTO-Full, a filtered US patent reaction dataset obtained by a text mining approach. In addition, the external server 20 may use the PubChem compound database including 111 million molecules and the ChEMBL database. For example, the external server 20 may be a database for prediction processing of the prediction system 10 or a server providing the database.

The prediction system 10 may include a control unit 11, a communication unit 12, an input/output interface unit 13, and a memory unit 14.

The control unit 11 is a configuration that controls the entire prediction system 10, and may include, for example, a processing unit such as a central processing unit (CPU) or a graphics processing unit (GPU). The control unit 11 may train models to be described below using information stored in the memory unit 14, and may also perform prediction value calculation for new inputs through the trained model. Specifically, the control unit 11 may control a retrosynthetic prediction model. To this end, the control unit 11 may include a control program such as an operating system (OS), a program that defines various processing orders, etc., and an internal memory for storing data. In addition, the control unit 11 may perform information processing for executing various processes by these programs, etc.

In addition, the communication unit 12 may include an interface that may be connected to a communication device such as a router connected to a communication line, etc., and may control communication between the prediction system 10 and the external server 20.

The input/output interface unit 13 may be an interface connected to an input unit 15 and/or a display unit 16. A user may communicate with the prediction system 10 through the input/output interface unit 13. For example, the display unit 16 may be a display means (for example, a display, monitor, touch panel, etc., composed of a liquid crystal display or an organic EL, etc.) that displays a display screen of an application, etc. In addition, the input unit 15 may be, for example, a key input unit, a touch panel, a control pad (for example, a touch pad, a game pad, etc.), a mouse, a keyboard, a microphone, etc.

In addition, the memory unit 14 may be a device that stores various databases or tables, etc. For example, the memory unit may include information on reactants and product pairs, information on the AE of molecules, etc. For example, the external server 20 may include a large corpus of information on various source-target pairs, and for this purpose, may use a subset of USPTO-Full, a filtered US patent reaction dataset obtained by a text mining approach. In addition, the external server 20 may also include the PubChem compound database including 111 million molecules and the ChEMBL database.

The embodiments according to the present invention described above are implemented in the form of program commands that can be executed through various computer components and recorded on a computer-readable recording medium. The computer-readable recording medium may include program commands, data files, data structures, etc., alone or in combination. The program commands recorded on the computer-readable recording medium may be especially designed and constituted for the present invention or may be known to those skilled in a field of computer software and available for use. Examples of the computer-readable recording medium may include a magnetic medium such as a hard disk, a floppy disk, and a magnetic tape, an optical recording medium such as a CD-ROM, a DVD; a magneto-optical medium such as a floptical disk, and a hardware device especially configured to store and perform program commands such as a ROM, a RAM, a flash memory, or the like. Examples of the program instructions include high-level language code capable of being executed by a computer using an interpreter, or the like, as well as machine language code created by a compiler. The hardware device may be configured to be operated as one or more software modules to perform processing according to the present invention, and vice versa.

In addition, the embodiments according to the present invention described above may be a set of program commands that can be executed through various computer components and a user application itself for executing the set of program commands. Specifically, it may be a program itself that may be downloaded through a server or a storage medium and installed on a client computer.

Hereinabove, although the present invention has been described with specific details such as specific components, limited examples, and drawings, they are provided only for assisting the overall understanding of the present invention. Therefore, the present invention is not limited to the exemplary embodiments. Various modifications and changes may be made by those skilled in the art to which the present invention pertains from this description.

Therefore, the spirit of the present invention should not be limited to the above-described exemplary embodiments, and not only the following claims but also modifications equivalent to the claims are intended to fall within the scope and spirit of the invention.

### INDUSTRIAL APPLICABILITY

The present invention can be used for the synthesis of various compounds including bio, pharmaceuticals, etc., and therefore can be used in industrial fields such as medicine, bioengineering, and chemistry.

1. A retrosynthetic translation method of predicting a reactant for a product using a neural machine translation (NMT) model based on transformer architecture, comprising:
preparing an input sequence and an output sequence of the model, in which the input sequence and the output sequence represent molecules as a list of fragments, each fragment constituting the list of fragments is a fragment expressed based on an atom environment (AE), and the product and the reactant are converted into a sequence expressed as the AE and prepared as the input sequence and the output sequence, respectively;
training the model using the input sequence and the output sequence; and
predicting the reactant by retro-synthesizing the product through the trained model, wherein a new product is converted into a sequence represented by the AE and input as an input sequence of the model, an output sequence is output through the model, and the predicted reactant is detected based on the output sequence,
wherein the AE is a fragment composed of a central atom having a predetermined radius and its covalent neighbors, and the predetermined radius is a maximum allowable topological distance between the central atom and all covalent atoms.

2. The retrosynthetic translation method of claim 1, wherein the predetermined radius is the number of bonds on a shortest pathway between atoms.

3. The retrosynthetic translation method of claim 2, wherein the fragment is expressed as one of a set of AEs (AE0) with a predetermined radius of 0 and a set of AEs (AE2) with a predetermined radius of 1.

4. The retrosynthetic translation method of claim 2, wherein the fragment is expressed as a combination of a set of AEs (AE0) with a predetermined radius of 0 and a set of AEs (AE2) with a predetermined radius of 1.

5. The retrosynthetic translation method of claim 1, wherein the AE is expressed as a simplified molecular-input line-entry system arbitrary target specification (SMARTS) pattern.

6. The retrosynthetic translation method of claim 5, wherein the SMARTS pattern for each of the AEs is associated with a unique integer value.

7. The retrosynthetic translation method of claim 1, wherein the AE is generated by an Extended Circular FingerPrint (ECFP) algorithm.

8. The retrosynthetic translation method of claim 1, wherein the model uses an encoder unit and a decoder unit, and applies a multi-head attention mechanism to each unit to translate the input sequence and the output sequence.

9. A retrosynthetic translation apparatus that predicts a reactant for a product using a neural machine translation (NMT) model based on transformer architecture, comprising:
a control unit configured to control the NMT model;
a communication unit configured to communicate with an external server;
a memory unit;
a display unit; and
an input unit configured to receive a user input,
wherein the memory unit includes an input sequence and an output sequence of the model, the input sequence and the output sequence represent molecules as a list of fragments, each fragment constituting the list of fragments is a fragment expressed based on an atom environment (AE), and the product and the reactant are converted into a sequence expressed as the AE and stored as the input sequence and the output sequence, respectively,
the control unit trains the model through the input sequence and the output sequence,
the control unit converts a new product into a sequence represented by the AE and inputs the sequence as an input sequence of the model, outputs an output sequence through the model, and detects a predicted reactant based on the output sequence, and
the AE is a fragment composed of a central atom having a predetermined radius and its covalent neighbors, and the predetermined radius is a maximum allowable topological distance between the central atom and all covalent atoms.

10. The retrosynthetic translation apparatus of claim 9, wherein the predetermined radius is the number of bonds on a shortest pathway between atoms.

11. The retrosynthetic translation apparatus of claim 10, wherein the fragment is expressed as one of a set of AEs (AE0) with a predetermined radius of 0 and a set of AEs (AE2) with a predetermined radius of 1.

12. The retrosynthetic translation apparatus of claim 10, wherein the fragment is expressed as a combination of a set of AEs (AE0) with a predetermined radius of 0 and a set of AEs (AE2) with a predetermined radius of 1.

13. The retrosynthetic translation apparatus of claim 9, wherein the AE is expressed as a simplified molecular-input line-entry system arbitrary target specification (SMARTS) pattern.

14. The retrosynthetic translation apparatus of claim 13, wherein the SMARTS pattern for each of the AEs is associated with a unique integer value.

15. The retrosynthetic translation apparatus of claim 9, wherein the AE is generated by an Extended Circular FingerPrint (ECFP) algorithm.

16. The retrosynthetic translation apparatus of claim 9, wherein the model uses an encoder unit and a decoder unit, and applies a multi-head attention mechanism to each unit to translate the input sequence and the output sequence.
